SEQUENCE LISTING (17 sequences)

SEQ ID NO: 1 - DNA, 1006 bp, Gallus gallus
ctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagctt

SEQ ID NO: 2 - DNA, 1272 bp, Artificial Sequence
  Description of Artificial Sequence Synthetic polynucleotide
ctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagctt

SEQ ID NO: 3 - DNA, 4324 bp, Artificial Sequence
  Description of Artificial Sequence Synthetic polynucleotide
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttcatcctgagcgctaatcgggtattgttcggttccatttaaccgaagaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 4 - DNA, 5925 bp, Artificial Sequence
  Description of Artificial Sequence Synthetic polynucleotide
tcgacatgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagccccctcactgcagattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattctgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttcatcctgagcgctaatcgggtattgttcggttccatttaaccgaagaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 5 - DNA, 5677 bp, Artificial Sequence
  Description of Artificial Sequence Synthetic polynucleotide
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgcgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtaggtttatatcttcccttctctgttcctccgcaggaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 6 - DNA, 6557 bp, Artificial Sequence
  Description of Artificial Sequence Synthetic polynucleotide
tcgactgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttctgcaggtcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttcatcctgagcgctaatcgggtattgttcggttccatttaaccgaagaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 7 - DNA, 4688 bp, Artificial Sequence
  Description of Artificial Sequence Synthetic polynucleotide
gtcgacgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaactctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 8 - DNA, 5695 bp, Artificial Sequence
  Description of Artificial Sequence Synthetic polynucleotide
gtcgacgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaactctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 9 - DNA, 6683 bp, Artificial Sequence
  Description of Artificial Sequence Synthetic polynucleotide
ggtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccagtcgacgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaactctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctctgcagtgactcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttgggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 10 - DNA, 4554 bp, Artificial Sequence
  Description of Artificial Sequence Synthetic polynucleotide
gtcgacgtgacgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcctgcagggatatcgaatttcgagggcccgtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccggggcgggctcaggggcgggctcaggggcggggcgggcgcgaaggtcctcccgaggcccggcattctcgcacgcttcaaaagcgcacgtctgccgcgctgttctcctcttcctctccggcctttcaagcttaccagcttgaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggccggtatcgatg

SEQ ID NO: 11 - DNA, 5882 bp, Artificial Sequence
  Description of Artificial Sequence Synthetic polynucleotide
gtcgactgggggctgcggaggaacagagaagggaagatataaaccccgccggcgccgacgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgacgtcgagctgcagcggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcactgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttcatcctgagcgctaatcgggtattgttcggttccatttaaccgaagaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 12 - DNA, 6022 bp, Artificial Sequence
  Description of Artificial Sequence Synthetic polynucleotide
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgcgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtaggtttatatcttcccttctctgttcctccgcaggaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctctctgcagtgggggctgcggaggaacagagaagggaagatataaaccccgccggcgccgacgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgacgtcgagctgcagcggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcactgcacaagcttgggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 13 - DNA, 1335 bp, Artificial Sequence
  Description of Artificial Sequence Synthetic polynucleotide
gggggctgcggaggaacagagaagggaagatataaaccccgccggcgccgacgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgacgtcgagctgcagcggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcactgca

SEQ ID NO: 14 - DNA, 1505 bp, Artificial Sequence
  Description of Artificial Sequence Synthetic polynucleotide
ccggaattcccaccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagcggccgcaaaaggaaaa

SEQ ID NO: 15 - DNA, 4590 bp, Gallus gallus
caccggtgttattgctgctcggtgcgtgcatgcacatcagtgtcgctgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcgtcggcgccggcggggtttatatcttcccttctctgttcctccgcagcccccaagcttcatcctgagcgctaatcgggtattgttcggttccatttaaccgaagaattcatgctagctctgttagccaatgcggccgcatagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 16 - DNA, 367 bp, Gallus gallus
ctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacag

SEQ ID NO: 17 - DNA, 938 bp, Artificial Sequence
  Description of Artificial Sequence Synthetic polynucleotide
acgcgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaactctgcagaaccaatgcattggat

(19) United States
(12) Patent Application Publication
(10) Pub. No.: US 2010/0216188 A1
(43) Pub. Date: Aug. 26, 2010

(54) USE OF CHICK BETA ACTIN GENE INTRON-1

(75) Inventor: Mizhou Hui, Thousand Oaks, CA (US)

Correspondence Address: OCCHIUTI ROHLICEK & TSAO, LLP, 10 FAWCETT STREET, CAMBRIDGE, MA, 02138, US

(73) Assignee: AMProtein Corporation, Camarillo, CA (US)

(21) Appl. No.: 12/524,347

(22) PCT Filed: Jun. 21, 2007
(86) PCT No.: PCT/US07/14488

§ 371 Date: Mar. 25, 2010

Related U.S. Application Data
(60) Provisional application No. 60/897,394, filed on Jan. 25, 2007.

(51) Int. Cl. C12P 21/06; C12N 15/63

(52) U.S. Cl. 435/69.1; 435/320.1

(57) ABSTRACT

A method to use chick beta actin gene intron-1 or functional equivalent as a gene expression enhancer element or a gene expression “hot spot” sequence for constructing or reconstructing a mammalian expression vector for extremely high expression of recombinant proteins is disclosed. Composition of a set of extremely strong gene expression vectors is also disclosed.

### RELATED APPLICATION

This application claims priority to U.S. Provisional Application Ser. No. 60/897,394, filed in Jan. 25, 2007, the content of which is incorporated herein by reference in its entirety.

### FIELD OF THE INVENTION

The present invention relates to use of chick beta actin gene Intron-1 as gene expression enhancer or a gene expression “hot spot” at 5′- or 3′-flanking region of a mammalian gene expression promoter to construct a new mammalian expression vector or reconstruct an existed gene expression vector for extremely high-level expression of recombinant proteins and generation of mammalian cell lines producing extremely high level of recombinant proteins.

### BACKGROUND OF THE INVENTION

A recombinant protein may be prepared by first introducing an expression vector encoding the recombinant protein into host cells and then express the recombinant protein in the host cells. Traditional host cells include original CHO, NSO and 293 cells not selected for optimal robust growth in serum-free suspension media. Traditional expression vectors may use SV40 or CMV based promoter to control the expression of the recombinant protein. The host cells employed in the conventional expression system grow relatively slow with double time of about 24-36 hours and optimal growing cell-density 3-5×10⁶ cells/ml.

To increase the production speed and maintain high production yield of recombinant proteins, the inventor finds that certain robust host cells with shorter double time and higher cell density may preferably be used. The robust cell lines are usually selected by screening fast and high-density growing cell lines or screened from any types of cell lines based on fast and high-density growth. However, promoters used in conventional expression vectors are not strong enough in these fast and high-density growing cell lines for high level of gene expression. In addition, not many vectors can be used universally to most types of cell lines.

Therefore, there is a need to search for extremely strong universal gene expression vectors that are suitable to be used in most of the robust fast growing host cells with shorter double time and high-density growth.

It was known that plant gene 5′ regulatory regions often contain high GC-rich content (CpG islands). Plant gene expression is often constitutive at higher level than that of mammalian expression. Probably, high GC-rich content with strong DNA structure at 5′ regulatory region plays a key role for all gene expression as a universal mechanism. Through genome DNA sequence research and previous laboratory experiences in the field, extremely high GC-rich content of chick beta actin gene intron-1 was identified (1.006 kb fragment, SEQ ID No:1). This 1006 base pair sequence contains average 74.8% GC content with the highest GC content 90.8% of a 130 base pair fragment. Through our experimental approach. We also found that this region has extremely strong DNA secondary structure, which was evidenced by great difficulty of sequencing, impossible for PCR reading through, and difficulty of ligation. We therefore hypothesized that genomic DNA of highly GC-rich with strong DNA structure might hold secret of high constitutive level of all mammalian gene expression through regulating chromatin condensation, and nucleosome-formation, which regulates gene transcription.

This invention is based on a surprising discovery, namely use of highly GC-rich chick beta actin gene Intron-1 as 5′- or/and 3′-flanking gene expression enhancer or gene expression “hot spot” site to construct a new mammalian expression vector or modify an existed vector for high-level expression of recombinant proteins. Surprisingly, the chick actin gene intron-1 modified mammalian expression vectors generated extremely high levels of gene expression in a fast-growing CHO Cell line.

In brief, chick beta actin intron-1 (1.006 kb fragment, SEQ ID No:1) was used as an enhancer element or an expression “hot spot” sequence and constructed around a given mammalian gene promoter and illustrated below:

1). Control (Actin promoter-ploy linker-polyA);

2). pMH1 (Intron-1-actin promoter-ploy linker-polyA);

3) pMH2 (Actin promoter-poly linker-polyA-Intron-1);

4). pMH3 (Intron1-actin promoter-poly linker-polyA-intron-1;

5) pMH4 (pCMV promoter-Intron1-poly linker-polyA);

6). pMH5 (pCMV promoter-Intron-1-poly linker-polyA-Intron-1);

7). pMH6 (pIntron-1-CMV promoter-Intron-1-poly linker-polyA-Intron-1);

8). pMH7 (pIntron-1-PGK promoter-poly linker-polyA);

9). pMH8 (pGC rich fragment-actin promoter-poly linker-polyA);

10). pMH9 (pActin promoter-poly linker-polyA-GC rich fragment);

### BRIEF SUMMARY OF THE INVENTION

A method to use chick beta actin intron-1 or its functional equivalent as an enhancer element or expression “hot spot” sequence for constructing extremely strong mammalian expression vector is disclosed. Composition of a set of extremely strong gene expression vectors is also disclosed.

### BRIEF DESCRIPTION OF THE DRAWINGS

FIG. 1A control plasmid of pActin Promoter-ploy linker-polyA is a native chick beta actin promoter-based expression vector. It was constructed by using 1.272 kb XhoI/HindIII fragment of the full length of chick beta-actin gene promoter (SEQ ID No:2) inserted to SalI/HindIII opened pBR322 vector backbone with EcoRI/NotI poly linker followed by a Poly A site.

FIG. 2 An intron-1 modified plasmid of pMH1 (Intron-1-actin promoter-ploy linker-polyA) (SEQ ID No:4) was constructed by inserting 1.006 kb of SalI/PstI adaptor modified Intron-1 to SalI/PstI sites immediately upstream of an action promoter sequence. Then, a 0.331 kb spacer fragment (CMV enhancer without CMV promoter) was inserted to PstI site in between Intron-1 and actin promoter at sense orientation.

FIG. 3 An intron-1 modified plasmid of pMH2 (Actin promoter-poly linker-polyA-Intron-1) (SEQ ID No:5) was constructed by inserting PstI/HindIII adaptor modified 1.006 kb intron-1 sequence to PstI/Hind III site immediately downstream of a Poly A signal sequence. Then, a 0.331 kb spacer fragment (CMV enhancer without CMV promoter) was inserted to PstI site in between Intron-1 and actin promoter at sense orientation.

FIG. 4 An Intron-1 modified plasmid of pMH3 (Intron1-actin promoter-poly linker-polyA-intron-1 (SEQ ID No:6) was constructed by combining PvuI/NotI fragments containing actin promoter of pMH1 (SEQ ID No:5) and PvuI/NotI fragments containing pBR322 backbone of pMH2 (SEQ ID No:4).

FIG. 5 An Intron-1 modified plasmid of pMH4 (pCMV promoter-Intron1-poly linker-polyA) (SEQ ID No:7) was constructed by combining a PCR amplified 0.82 kb CMV promoter sequence with SalI/PstI sites and PstI/HindIII modified intron-1 fragment together. It was then inserted to SalI/Hind III site of SalI/HindIII opened pBR322 vector backbone with EcoRI/NotI linker followed by a Poly A site.

FIG. 6 An Intron-1 modified plasmid of pMH5 (pCMV promoter-Intron-1-poly linker-polyA-Intron-1) (SEQ ID No:8) was constructed by combining PvuI/NotI fragments containing actin promoter of pMH4 (SEQ ID No:7) and PvuI/NotI fragments containing pBR322 backbone of pMH2 (SEQ ID No:5).

FIG. 7 An Intron-1 modified plasmid of pMH6 (pIntron-1-CMV promoter-Intron-1-poly linker-polyA-Intron-1) (SEQ ID No:9) was constructed by inserting SalI modified 1.006 kb intron-1 sequence to SalI site immediately upstream of a CMV promoter of pMH5 (pCMV promoter-Intron-1-poly linker-polyA-Intron-1) at sense orientation.

FIG. 8 An Intron-1 modified plasmid of pMH7 (pIntron-1-PGK promoter-poly linker-polyA) (SEQ ID No:10) was constructed by inserting 0.572 kb PCR amplified PGK promoter sequence with PstI/HindIII sites to PstI/HindIII opened pBR322 vector backbone with EcoRI/NotI linker followed by a Poly A site. An Intron-1 sequence with adaptor modified SalI/PstI sites was then inserted to SalI/PstI sites immediately upstream of PGK promoter.

FIG. 9 A GC-rich DNA fragment modified plasmid of pMH8 (pGC rich fragment-actin promoter-poly linker-polyA) (SEQ ID No:11) was constructed by inserting a synthetic 1.337 kb GC-rich fragment (SEQ ID No:13) with SalI/PstI sites to SalI/PstI sites immediately upstream of an actin promoter sequence of pBR322 vector backbone with EcoRI/NotI linker followed by a Poly A site.

FIG. 10 A GC-rich DNA fragment modified plasmid of pMH9 (pActin promoter-poly linker-polyA-GC-rich fragment) (SEQ ID No:12) was constructed by inserting the PstI/HindIII adaptor modified synthetic 1.337 kb GC-rich fragment (SEQ ID No:13) to PstI/HindIII sites downstream of a Poly A signal sequence.

### DETAILED DESCRIPTION OF THE INVENTION

This invention is based on discovery of use of chick beta actin gene Intron-1 as an enhancer element or an expression “hot spot” sequence to construct mammalian expression vector for extremely high-level expression of recombinant proteins. In brief, chick beta actin gene intron-1 (1.006 kb fragment SEQ No:1) was used as an enhancer sequence or hot spot and constructed around a given mammalian gene promoter and illustrated below:

1). Control (Actin promoter-ploy linker-polyA);

2). pMH1 (Intron-1-actin promoter-ploy linker-polyA);

3) pMH2 (Actin promoter-poly linker-polyA-Intron-1);

4). pMH3 (Intron1-actin promoter-poly linker-polyA-intron-1;

5) pMH4 (pCMV promoter-Intron1-poly linker-polyA);

6). pMH5 (pCMV promoter-Intron-1-poly linker-polyA-Intron-1);

7). pMH6 (pIntron-1-CMV promoter-Intron-1-poly linker-polyA-Intron-1);

8). pMH7 (pIntron-1-PGK promoter-poly linker-polyA);

9). pMH8 (pGC rich fragment-actin promoter-poly linker-polyA);

10). pMH9 (pActin promoter-poly linker-polyA-GC rich fragment);

Full length of chick beta actin gene 5′-flanking regulatory element was from Dr. N Fregien (ATCC 37507) (Fregien N and Davidson N, 1986). It was sequenced and characterized by restriction enzyme mapping and matched to the sequence published (Kost et al., 1983). A 1.494 kb chick actin gene promoter fragment was digested by Pst I and Hind III and purified by SDS gel. This 1.494 kb Pst I/Hind III promoter fragment was further digested by Hinfl to obtain 1:006 kb Intron-1 and modified by using a phosphorylated Pst I/Hinfl adaptor to have Pst I at 5′-end and Hind III at 3′-end of the intron-1 (SEQ No:1).

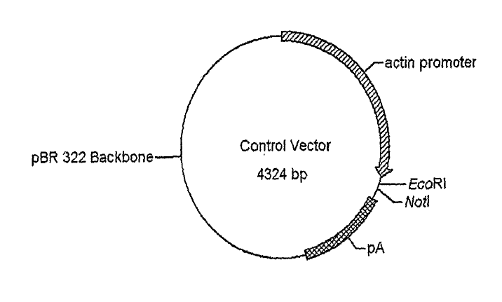

The native chick beta actin promoter-based expression vector (FIG. 1) (SEQ ID NO: 3) was constructed by inserting a 1.272 kb Xho I/Hind III fragment of full length of chick beta actin gene 5′-flanking regulatory element containing, intron-1 (SEQ ID No:2) into a SalI/HindIII opened pBR322-based vector backbone with EcoRI/NotI sites followed by a poly A site to form Control (Actin promoter-ploy linker-polyA) (SEQ ID NO: 3).

A control plasmid of pActin Promoter-ploy linker-polyA (FIG. 1) is a native chick beta actin promoter-based expression vector. It was constructed by using 1.272 kb XhoI/HindIII fragment of the full length of chick beta-actin gene promoter (SEQ ID No:2) inserted to SalI/HindIII opened pBR322 vector backbone with EcoRI/NotI poly linker followed by a Poly A site.

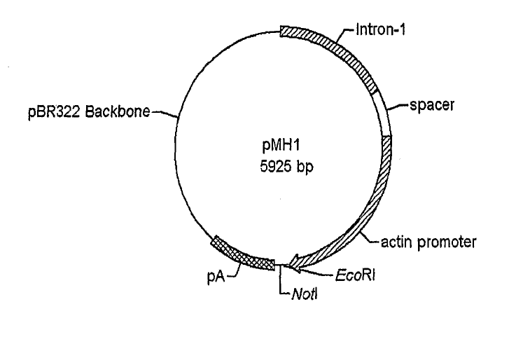

An intron-1 modified plasmid of pMH1 (Intron-1-actin promoter-ploy linker-poly A) (FIG. 2) (SEQ ID No:4) was constructed by inserting 1.006 kb of SalI/PstI adaptor modified Intron-1 to SalI/PstI sites immediately upstream of an action promoter sequence. Then, a 0.331 kb spacer fragment (CMV enhancer without CMV promoter) was inserted to PstI site in between Intron-1 and actin promoter at sense orientation.

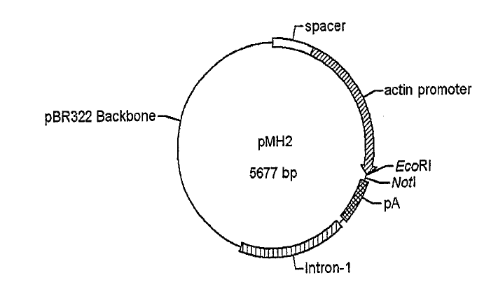

An intron-1 modified plasmid of pMH2 (Actin promoter-poly linker-poly A-Intron-1) (FIG. 3) (SEQ ID No:5) was constructed by inserting PstI/HindIII adaptor modified 1.006 kb intron-1 sequence to PstI/Hind III site immediately downstream of a Poly A signal sequence. Then, a 0.331 kb spacer fragment (CMV enhancer without CMV promoter) was inserted to PstI site in between Intron-1 and actin promoter at sense orientation.

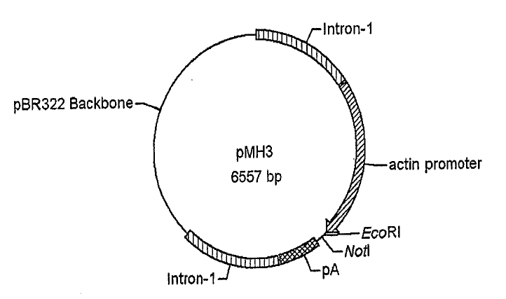

An Intron-1 modified plasmid of pMH3 (Intron1-actin promoter-poly linker-polyA-intron-1) (FIG. 4) (SEQ ID No:6) was constructed by combining PvuI/NotI fragments containing actin promoter of pMH1 (SEQ ID No:5) and PvuI/NotI fragments containing pBR322 backbone of pMH2 (SEQ ID No:4).

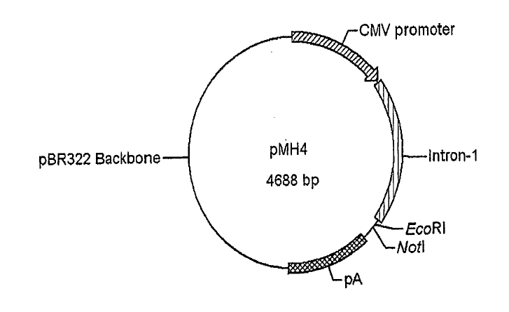

An Intron-1 modified plasmid of pMH4 (pCMV promoter-Intron1-poly linker-polyA) (FIG. 5) (SEQ ID No:7) was constructed by combining a PCR amplified 0.82 kb CMV promoter sequence with SalI/PstI sites and PstI/HindIII modified intron-1 fragment together. It was then inserted to SalI/Hind III site of SalI/HindIII opened pBR322 vector backbone with EcoRI/NotI linker followed by a Poly A site.

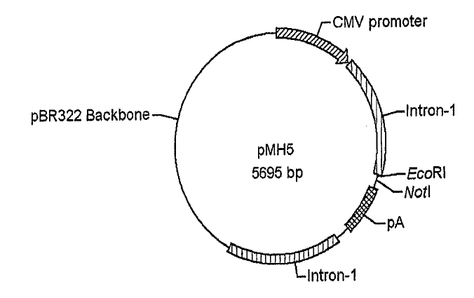

An Intron-1 modified plasmid of pMH5 (pCMV promoter-Intron-1-poly linker-polyA-Intron-1) (FIG. 6) (SEQ ID No:8) was constructed by combining PvuI/NotI fragments containing actin promoter of pMH4 (SEQ ID No:7) and PvuI/NotI fragments containing pBR322 backbone of pMH2 (SEQ ID No:5).

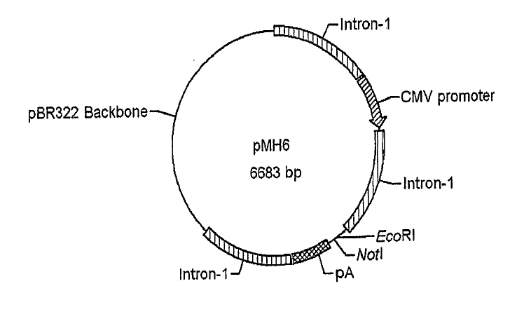

An Intron-1 modified plasmid of pMH6 (pIntron-1-CMV promoter-Intron-1-poly linker-polyA-Intron-1) (FIG. 7) (SEQ ID No:9) was constructed by inserting SalI modified 1.006 kb intron-1 sequence to SalI site immediately upstream of a CMV promoter of pMH5 (pCMV promoter-Intron-1-poly linker-polyA-Intron-1) at sense orientation.

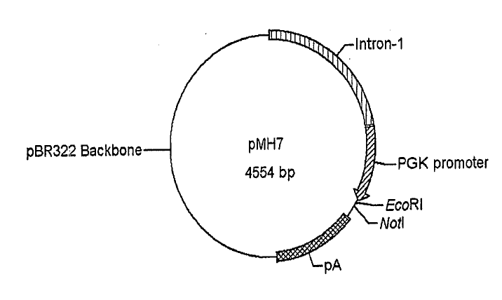

An Intron-1 modified plasmid of pMH7 (pIntron-1-PGK promoter-poly linker-polyA) (FIG. 8) (SEQ ID No:10) was constructed by inserting 0.572 kb PCR amplified PGK promoter sequence with PstI/HindIII sites to PstI/HindIII opened pBR322 vector backbone with EcoRI/NotI linker followed by a Poly A site. An Intron-1 sequence with adaptor modified SalI/PstI sites was then inserted to SalI/PstI sites immediately upstream of PGK promoter.

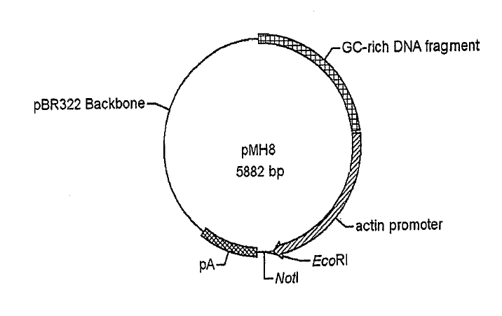

A GC-rich DNA fragment (SEQ ID No:13) modified plasmid of pMH8 (pGC rich fragment-actin promoter-poly linker-polyA) (FIG. 9) (SEQ ID No:11) was constructed by inserting a synthetic 1.337 kb GC-rich fragment (SEQ ID No:13) with SalI/PstI sites to SalI/PstI sites immediately upstream of an actin promoter sequence of pBR322 vector backbone with EcoRI/NotI linker followed by a Poly A site.

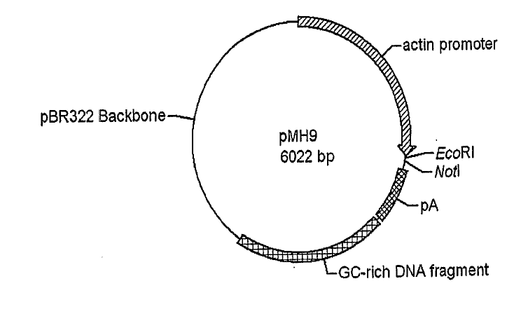

A GC-rich DNA fragment (SEQ ID No 13) modified plasmid of pMH9 (pActin promoter-poly linker-polyA-GC-rich fragment) (FIG. 10) (SEQ ID No:12) was constructed by inserting the PstI/HindIII adaptor modified synthetic 1.337 kb GC-rich fragment (SEQ ID No:13) to PstI/HindIII sites downstream of a Poly A signal sequence.

A cDNA encoding EcoRI site-TNFR2-Fc-Not I site (SEQ ID No 14) was removed form a previous plasmid vector (in house) and inserted into EcoRI/Not I sites of the above constructed mammalian expression vectors shown in FIG. 1-10 (SEQ ID No 3, 4, 5, 6, 7, 8, 9, 10, 11, 12). These plasmid cDNAs were linearized fby PvuI and stably transfected into a fast growing CHO parental host line using a Gene Pulser (Bio-Rad). PGK promoter driven neomycin resistant gene was used for stable cell clone selection either through co-transfection or through inserting PGK-Neo resistant gene-pA cassette into SalI site of the each vector.

The stable cell clones were picked into a 96-well plate (NUNC). The transfection was repeated. All gene expressions were conducted in 0.1 ml freshly added serum-free medium at 37 C in a CO2 incubator in 96-well plate for 3 hours.

The TNFR2-Fc expression of 3 hours in fresh serum-free medium was detected by using a dot-blot or Elisa. Anti-IgG1 Fc fragment antibodies conjugated with HRP (PIERCE) were used for the specific binding. Expression titer of the best clone from the above two transfections of 2×96-well plates was used to compare expression titer of each constructs.

In brief, the harvested conditional media were diluted seriously at 0, 2, 4, 8, 16, and 32 times. The diluted conditional media were subjected to dot blot semi-quantitative assay using anti human Ig Fc antisera conjugated with HRP (PIERCE). Alternatively, 96-well microplate for a standard Elisa was coated by using 0.1 ml of the diluted conditional media followed by incubating with anti human Ig Fc antisera conjugated with HRP (PIERCE), washing, color development and quantitation by a microplate reader. Commercial available TNFR2-Fc (Enbrel) was added to our serum-free culture medium and used as a quantitative standard.

TABLE 1

| | | # of | Expression titer |
|---|---|---|---|
| | | clones | (pg/cell/day) of |
| Vector | Figure/SEQ ID | screened | the best clone |
| Control | FIG. 1/(SEQ ID No: 3 | 96 × 2 | 7 ± 2 |
| pMH1 | FIG. 2/SEQ ID No: 4 | 96 × 2 | 53 ± 4 |
| pMH2 | FIG. 3/SEQ ID No: 5 | 96 × 2 | 52 ± 4 |
| pMH3 | FIG. 4/SEQ ID No: 6 | 96 × 2 | 67 ± 5 |
| pMH4 | FIG. 5/SEQ ID No: 7 | 96 × 2 | 56 ± 3 |
| pMH5 | FIG. 6/SEQ ID No: 8 | 96 × 2 | 60 ± 5 |
| pMH6 | FIG. 7/SEQ ID No: 9 | 96 × 2 | 69 ± 7 |
| pMH7 | FIG. 8/SEQ ID No: 10 | 96 × 2 | 45 ± 2 |
| pMH8 | FIG. 9/SEQ ID No: 11 | 96 × 2 | 41 ± 4 |
| pMH9 | FIG. 10/SEQ ID No: 12 | 96 × 2 | 39 ± 5 |

The results in Table 1 indicated that this 1.006 kb chick beta actin gene Intron-1 could be used as a common gene expression enhancer element or gene expression “hot spot” sequence at 5′- or 3′-flanking of a mammalian gene expression promoter to construct a new mammalian expression vector or reconstruct an existed gene expression vector for high-level expression of recombinant proteins and generation of mammalian cell lines producing high level of recombinant proteins. The results also showed that it is not only an enhancer element but also a “hot spot” sequence since it works well at all different locations of the expression vectors. In addition, it showed that a synthetic GC-rich fragment also can be used as a common gene expression enhancer element or gene expression “hot spot” sequence at 5′- or 3′-flanking of a mammalian gene expression promoter. All the expression titers reached or exceeded high end of current industrial levels (15-45 pg/cell/day), suggesting great commercial value of these expression vectors. We believed that we had solved mammalian gene expression once for all and identified probably a common method or mechanism of all gene expression, namely use of naturally occurred or synthetic GC-rich DNAs with strong secondary structure as enhancers or expression “hot spot” sequences for high constitutive mammalian gene expression.

As we discussed earlier in this invention, plant gene 5′ regulatory regions often contain high GC-rich content called CpG islands. Plant gene expression is often constitutive at higher levels. The results in Table 1 indicated that a naturally occurred intron-1 of chick beta actin gene with extremely high GC-rich content and possible strong DNA structure played a key role for CHO cell gene expression. This indicated that searching for high GC content introns or expression enhancer or insulators for eukaryotic gene expression will be a universal tool for constructing or reconstructing effective gene expression vectors. Other option is to synthesize artificial GC-rich introns, “hot spot”, enhancers, promoters for constructing and reconstructing effective gene expression vectors by following this common mechanism.

The results in Table 1 also indicated that integration of non-specific synthetic DNA fragments with high GC content and possible strong DNA structure support high level of constitutive gene expression in CHO cells, suggesting future synthetic or modified gene expression enhancer or “hot spot” sequences as a universal tool for gene expression vector construction. We concluded that high GC-rich DNA sequence could be used to construct to reconstruct gene expression vectors as a common method for high gene expression. Very likely, high GC-content DNA fragment with strong DNA structure is a universal mechanism that regulates chromatin condensation and nucleosome-formation for high level of gene transcription and expression.

By the terminology “GC-rich fragment” as used throughout this description (unless otherwise specified), there is meant a piece of DNA (100-2000 bp in length), either naturally occurring or synthesized, in which not less than about sixty eight percent (68%) by number of the bases are composed of cytosine (C) and/or guanine (G), and most preferably, eighty percent (80%) or more by number are composed of cytosine and/or guanine.

### Example 1

### Sequencing the 5′-Flanking Region of Chick Beta Actin Gene

5′-flanking region of chick beta actin gene was from Dr. N Fregien (ATCC 37507) (Fregien N and Davidson N, 1986) and sequenced by commercial service provider Laragen Inc. Complete sequence is listed below:

CACCGGTGTTATTGCTGCTCGGTGCGTGCATGCACATCAGTGTCGCT
GCAGCTCAGTGCATGCACGCTCATTGCCCATCGCTATCCCTGCCTCT
CCTGCTGGCGCTCCCCGGGAGGTGACTTCAAGGGGACCGCAGGACCA
CCTCGGGGGTGGGGGGAGGGCTGCACACGCGGACCCCGCTCCCCCTC
CCCAACAAAGCACTGTGGAATCAAAAAGGGGGGAGGGGGGATGGAGG
GGCGCGTCACACCCCCGCCCCACACCCTCACCTCGAGGTGAGCCCCA
CGTTCTGCTTCACTCTCCCCATCTCCCCCCCCTCCCCACCCCCAATT
TTGTATTTATTTATTTTTTAATTATTTTGTGCAGCGATGGGGGCGGG
GGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCT
ATAAAAAGCGAAGCGCGCGGCGGGCGGGAGTCGCTGCGTTGCCTTCG
CCCCGTGCCCCGCTCCGCGCCGCCTCGCGCCGCCCGCCCCGGCTCTG
ACTGACCGCGTTACTCCCACAGGTGAGCGGGCGGGACGGCCCTTCTC
CTCCGGGCTGTAATTAGCGCTTGGTTTAATGACGGCTCGTTTCTTTT
CTGTGGCTGCGTGAAAGCCTTAAAGGGCTCCGGGAGGGCCCTTTGTG
CGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGG
CGCGGCGCGGGGCTTTGTGCGCTCCGCGTGTGCGCGAGGGGAGCGCG
GCCGGGGGCGGTGCCCCGCGGTGCGGGGGGGCTGCGAGGGGAACAAA
GGCTGCGTGCGGGGTGTGTGCGTGGGGGGGTGAGCAGGGGGTGTGGG
CGCGGCGGTCGGGCTGTAACCCCCCCCTGCACCCCCCTCCCCGAGTT
GCTGAGCACGGCCCGGCTTCGGGTGCGGGGCTCCGTGCGGGGCGTGG
CGCGGGGCTCGCCGTGCCGGGCGGGGGGTGGCGGCAGGTGGGGGTGC
CGGGCGGGGCGGGGCCGCCTCGGGCCGGGGAGGGCTCGGGGGAGGGG
CGCGGCGGCCCCGGAGCGCCGGCGGCTGTCGAGGCGCGGCGAGCCGC
AGCCATTGCCTTTTATGGTAATCGTGCGAGAGGGCGCAGGGACTTCC
TTTGTCCCAAATCTGGCGGAGCCGAAATCTGGGAGGCGCCGCCGCAC
CCCCTCTAGCGGGCGCGGGCGAAGCGGTGCGGCGCCGGCAGGAAGGA
AATGGGCGGGGAGGGCCTTCGTGCGTCGCCGCGCCGCCGTCCCCTTC
TCCATCTCCAGCCTCGGGGCTGCCGCAGGGGGACGGCTGCCTTCGGG
GGGGACGGGGCAGGGCGGGGTTCGTCGGCGCCGGCGGGGTTTATATC
TTCCCTTCTCTGTTCCTCCGCAGCCCCCAAGCTTCATCCTGAGCGCT
AATCGGGTATTGTTCGGTTCCATTTAACCGAAGAATTCATGCTAGCT
CTGTTAGCCAATGCGGCCGCATAGATCTTTTTCCCTCTGCCAAAAAT
TATGGGGACATCATGAAGCCCCTTGAGCATCTGACTTCTGGCTAATA
AAGGAAATTTATTTTCATTGCAATAGTGTGTTGGAATTTTTTGTGTC
TCTCACTCGGAAGGACATATGGGAGGGCAAATCATTTAAAACATCAG
AATGAGTATTTGGTTTAGAGTTTGGCAACATATGCCCATATGCTGGC
TGCCATGAACAAAGGTTGGCTATAAAGAGGTCATCAGTATATGAAAC
AGCCCCCTGCTGTCCATTCCTTATTCCATAGAAAAGCCTTGACTTGA
GGTTAGTTTTTTTTATATTTTGTTTTGTGTTATTTTTTTCTTTAACA
TCCCTAAAATTTTCCTTACATGTTTTACTAGCCAGATTTTTCCTCCT
CTCCTGACTACTCCCAGTCATAGCTGTCCCTCTTCTCTTATGGAGAT
CCCTCGACCTGGCGTAATCATGGTCATAGCTGTTTCCTGTGTGAAAT
TGTTATCCGCTCACAATTCCACACAACATACGAGCCGGAAGCATAAA
GTGTAAAGCCTGGGGTGCCTAATGAGTGAGCTAACTCACATTAATTG
CGTTGCGCTCACTGCCCGCTTTCCAGTCGGGAAACCTGTCGTGCCAG
CGGATCCGCATCTCAATTAGTCAGCAACCATAGTCCCGCCCCTAACT
CCGCCCATCCCGCCCCTAACTCCGCCCAGTTCCGCCCATTCTCCGCC
CCATGGCTGACTAATTTTTTTTATTTATGCAGAGGCCGAGGCCGCCT
CGGCCTCTGAGCTATTCCAGAAGTAGTGAGGAGGCTTTTTTGGAGGC
CTAGGCTTTTGCAAAAAGCTAACTTGTTTATTGCAGCTTATAATGGT
TACAAATAAAGCAATAGCATCACAAATTTCACAAATAAAGCATTTTT
TTCACTGCATTCTAGTTGTGGTTTGTCCAAACTCATCAATGTATCTT
ATCATGTCTGGATCCGCTGCATTAATGAATCGGCCAACGCGCGGGGA
GAGGCGGTTTGCGTATTGGGCGCTCTTCCGCTTCCTCGCTCACTGAC
TCGCTGCGCTCGGTCGTTCGGCTGCGGCGAGCGGTATCAGCTCACTC
AAAGGCGGTAATACGGTTATCCACAGAATCAGGGGATAACGCAGGAA
AGAACATGTGAGCAAAAGGCCAGCAAAAGGCCAGGAACCGTAAAAAG
GCCGCGTTGCTGGCGTTTTTCCATAGGCTCCGCCCCCCTGACGAGCA
TCACAAAAATCGACGCTCAAGTCAGAGGTGGCGAAACCCGACAGGAC
TATAAAGATACCAGGCGTTTCCCCCTGGAAGCTCCCTCGTGCGCTCT
CCTGTTCCGACCCTGCCGCTTACCGGATACCTGTCCGCCTTTCTCCC
TTCGGGAAGCGTGGCGCTTTCTCATAGCTCACGCTGTAGGTATCTCA
GTTCGGTGTAGGTCGTTCGCTCCAAGCTGGGCTGTGTGCACGAACCC
CCCGTTCAGCCCGACCGCTGCGCCTTATCCGGTAACTATCGTCTTGA
GTCCAACCCGGTAAGACACGACTTATCGCCACTGGCAGCAGCCACTG
GTAACAGGATTAGCAGAGCGAGGTATGTAGGCGGTGCTACAGAGTTC
TTGAAGTGGTGGCCTAACTACGGCTACACTAGAAGAACAGTATTTGG
TATCTGCGCTCTGCTGAAGCCAGTTACCTTCGGAAAAAGAGTTGGTA
GCTCTTGATCCGGCAAACAAACCACCGCTGGTAGCGGTGGTTTTTTT
GTTTGCAAGCAGCAGATTACGCGCAGAAAAAAAGGATCTCAAGAAGA
TCCTTTGATCTTTTCTACGGGGTCTGACGCTCAGTGGAACGAAAACT
CACGTTAAGGGATTTTGGTCATGAGATTATCAAAAAGGATCTTCACC
TAGATCCTTTTAAATTAAAAATGAAGTTTTAAATCAATCTAAAGTAT
ATATGAGTAAACTTGGTCTGACAGTTACCAATGCTTAATCAGTGAGG
CACCTATCTCAGCGATCTGTCTATTTCGTTCATCCATAGTTGCCTGA
CTCCCCGTCGTGTAGATAACTACGATACGGGAGGGCTTACCATCTGG
CCCCAGTGCTGCAATGATACCGCGAGACCCACGCTCACCGGCTCCAG
ATTTATCAGCAATAAACCAGCCAGCCGGAAGGGCCGAGCGCAGAAGT
GGTCCTGCAACTTTATCCGCCTCCATCCAGTCTATTAATTGTTGCCG
GGAAGCTAGAGTAAGTAGTTCGCCAGTTAATAGTTTGCGCAACGTTG
TTGCCATTGCTACAGGCATCGTGGTGTCACGCTCGTCGTTTGGTATG
GCTTCATTCAGCTCCGGTTCCCAACGATCAAGGCGAGTTACATGATC
CCCCATGTTGTGCAAAAAAGCGGTTAGCTCCTTCGGTCCTCCGATCG
TTGTCAGAAGTAAGTTGGCCGCAGTGTTATCACTCATGGTTATGGCA
GCACTGCATAATTCTCTTACTGTCATGCCATCCGTAAGATGCTTTTC
CTGTGACTGGTGAGTACTCAACCAAGTCATTTGAGAATAGTGTATGC
GGCGACCGAGTTGCTCTTGCCCGGCGTCAATACGGGATAATACCGCG
CCACATAGCAGAACTTTAAAAGTGCTCATCATTGGAAAACGTTCTTC
GGGGCGAAAACTCTCAAGGATCTTACCGCTGTTGAGATCCAGTTCGA
TGTAACCCACTCGTGCACCCAACTGATCTTCAGCATCTTTTACTTTC
ACCAGCGTTTCTGGGTGAGCAAAAACAGGAAGGCAAAATGCCGCAAA
AAAGGGAATAAGGGCGACACGGAAATGTTGAATACTCATACTCTTCC
TTTTTCAATATTATTGAAGCATTTATCAGGGTTATTGTCTCATGAGC
GGATACATATTTGAATGTATTTAGAAAAATAAACAAATAGGGGTTCC
GCGCACATTTCCCCGAAAAGTGCCACCTGG

### Example 2

### Construction of Mammalian Expression Vectors

Full length of chick beta actin gene 5′-flanking regulatory element was from Dr. N Fregien (ATCC 37507) (Fregien N and Davidson N, 1986). It was sequenced and characterized by restriction enzyme mapping and matched to the sequence published (Kost et al., 1983). A 1.494 kb chick actin gene promoter fragment was digested by Pst I and Hind III and purified by SDS gel. This 1.494 kb Pst I/Hind III promoter fragment was further digested by Hinfl to obtain 1.006 kb Intron-1 and modified by using a phosphorylated Pst I/Hinfl adaptor to have Pst I at 5′-end and Hind III at 3′-end of the intron-1 (SEQ No:1).

The native chick beta actin promoter-based expression vector (FIG. 1) (SEQ ID NO: 3) was constructed by inserting a 1.272 kb Xho I/Hind III fragment of full length of chick beta actin gene 5′-flanking regulatory element containing intron-1 (SEQ ID No:2) into a SalI/HindIII opened pBR322-based vector backbone with EcoRI/NotI sites followed by a poly A site to form Control (Actin promoter-ploy linker-polyA) (SEQ ID NO: 3).

A control plasmid of pActin Promoter-ploy linker-polyA (FIG. 1) is a native chick beta actin promoter-based expression vector. It was constructed by using 1.272 kb XhoI/HindIII fragment of the full length of chick beta-actin gene promoter (SEQ ID No:2) inserted to SalI/HindIII opened pBR322 vector backbone with EcoRI/NotI poly linker followed by a Poly A site.

An intron-1 modified plasmid of pMH1 (Intron-1-actin promoter-ploy linker-poly A) (FIG. 2) (SEQ ID No:4) was constructed by inserting 1.006 kb of SalI/PstI adaptor modified Intron-1 to SalI/PstI sites immediately upstream of an action promoter sequence. Then, a 0.331 kb spacer fragment (CMV enhancer without CMV promoter) was inserted to PstI site in between Intron-1 and actin promoter at sense orientation.

An intron-1 modified plasmid of pMH2 (Actin promoter-poly linker-poly A-Intron-1) (FIG. 3) (SEQ ID No:5) was constructed by inserting PstI/HindIII adaptor modified 1.006 kb intron-1 sequence to PstI/Hind III site immediately downstream of a Poly A signal sequence. Then, a 0.331 kb spacer fragment (CMV enhancer without CMV promoter) was inserted to PstI site in between Intron-1 and actin promoter at sense orientation.

An Intron-1 modified plasmid of pMH3 (Intron1-actin promoter-poly linker-polyA-intron-1) (FIG. 4) (SEQ ID No:6) was constructed by combining PvuI/NotI fragments containing actin promoter of pMH1 (SEQ ID No:5) and PvuI/NotI fragments containing pBR322 backbone of pMH2 (SEQ ID No:4).

An Intron-1 modified plasmid of pMH4 (pCMV promoter-Intron1-poly linker-polyA) (FIG. 5) (SEQ ID No:7) was constructed by combining a PCR amplified 0.82 kb CMV promoter sequence with SalI/PstI sites and PstI/HindIII modified intron-1 fragment together. It was then inserted to SalI/Hind III site of SalI/HindIII opened pBR322 vector backbone with EcoRI/NotI linker followed by a Poly A site.

An Intron-1 modified plasmid of pMH5 (pCMV promoter-Intron-1-poly linker-polyA-Intron-1) (FIG. 6) (SEQ ID No:8) was constructed by combining PvuI/NotI fragments containing actin promoter of pMH4 (SEQ ID No:7) and PvuI/NotI fragments containing pBR322 backbone of pMH2 (SEQ ID No:5).

An Intron-1 modified plasmid of pMH6 (pIntron-1-CMV promoter-Intron-1-poly linker-polyA-Intron-1) (FIG. 7) (SEQ ID No:9) was constructed by inserting SalI modified 1.006 kb intron-1 sequence to SalI site immediately upstream of a CMV promoter of pMH5 (pCMV promoter-Intron-1-poly linker-polyA-Intron-1) at sense orientation.

An Intron-1 modified plasmid of pMH7 (pIntron-1-PGK promoter-poly linker-polyA) (FIG. 8) (SEQ ID No:10) was constructed by inserting 0.572 kb PCR amplified PGK promoter sequence with PstI/HindIII sites to PstI/HindIII opened pBR322 vector backbone with EcoRI/NotI linker followed by a Poly A site. An Intron-1 sequence with adaptor modified SalI/PstI sites was then inserted to SalI/PstI sites immediately upstream of PGK promoter.

A GC-rich DNA fragment (SEQ ID No:13) modified plasmid of pMH8 (pGC rich fragment-actin promoter-poly linker-polyA) (FIG. 9) (SEQ ID No:11) was constructed by inserting a synthetic 1.337 kb GC-rich fragment (SEQ ID No:13) with SalI/PstI sites to SalI/PstI sites immediately upstream of an actin promoter sequence of pBR322 vector backbone with EcoRI/NotI linker followed by a Poly A site.

A GC-rich DNA fragment (SEQ ID No 13) modified plasmid of pMH9 (pActin promoter-poly linker-polyA-GC-rich fragment) (FIG. 10) (SEQ ID No:12) was constructed by inserting the PstI/HindIII adaptor modified synthetic 1,337 kb GC-rich fragment (SEQ ID No:13) to PstI/HindIII sites downstream of a Poly A signal sequence.

### Example 3

### GC Content Analysis of Chick Beta Actin Gene Intron-1

Chick beta actin gene intron-1 (SEQ ID No:1) is listed below:

CTGCAGTGACTCGAGTCGCTGCGTTGCCTTCGCCCCGTGCCCCGCTC
CGCGCCGCCTCGCGCCGCCCGCCCCGGCTCTGACTGACCGCGTTACT
CCCACAGGTGAGCGGGCGGGACGGCCCTTCTCCTCCGGGCTGTAATT
AGCGCTTGGTTTAATGACGGCTCGTTTCTTTTCTGTGGCTGCGTGAA
AGCCTTAAAGGGCTCCGGGAGGGCCCTTTGTGCGGGGGGGAGCGGCT
CGGGGGGTGCGTGCGTGTGTGTGTGCGTGGGGAGCGCCGCGTGCGGC
CCGCGCTGCCCGGCGGCTGTGAGCGCTGCGGGCGCGGCGCGGGGCTT
TGTGCGCTCCGCGTGTGCGCGAGGGGAGCGCGGCCGGGGGCGGTGCC
CCGCGGTGCGGGGGGGCTGCGAGGGGAACAAAGGCTGCGTGCGGGGT
GTGTGCGTGGGGGGGTGAGCAGGGGGTGTGGGCGCGGCGGTCGGGCT
GTAACCCCCCCCTGCACCCCCCTCCCCGAGTTGCTGAGCACGGCCCG
GCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGC
CGCCTCGGGCCGGGGAGGGCTCGGGGGAGGGGCGCGGCGGCCCCGGA
GCGCCGGCGGCTGTCGAGGCGCGGCGAGCCGCAGCCATTGCCTTTTA
TGGTAATCGTGCGAGAGGGCGCAGGGACTTCCTTTGTCCCAAATCTG
GCGGAGCCGAAATCTGGGAGGCGCCGCCGCACCCCCTCTAGCGGGCG
CGGGCGAAGCGGTGCGGCGCCGGCAGGAAGGAAATGGGCGGGGAGGG
CCTTCGTGCGTCGCCGCGCCGCCGTCCCCTTCTCCATCTCCAGCCTC
GGGGCTGCCGCAGGGGGACGGCTGCCTTCGGGGGGGACGGGGCAGGG
CGGGGTTCGTCGGCGCCGGCGGGGTTTATATCTTCCCTTCTCTGTTC
CTCCGCAGCCCCCAAGCTT

High GC content regions of chick beta actin gene intron-1 was analyzed and summarized in Table 2 below.

TABLE 2

| Positions | 1-100 | 200-300 | 330-430 | 520-650 | 750-830 |
|---|---|---|---|---|---|
| GC content | 78.0% | 82.0% | 80.0% | 90.8% | 80.0% |

Extremely high GC content up to 90.8% was identified in the intron-1 with minimum DNA length of 100 base pair. This extremely high GC content is unusual in mammalian genome. How this had occurred through evolution in chick genome is unknown. Through experimental approach, we found that this region has extremely strong DNA secondary structure, which was evidenced by great difficulty of sequencing, impossible for PCR reading through, and difficulty of ligation. We hypothesized that genomic DNA of highly GC-rich with strong DNA structure might hold secret of high constitutive level of all mammalian gene expression through regulating chromatin condensation, and nucleosome-formation, which regulates gene transcription. We then synthesized a non-specific high GC content 1337 base pair DNA fragment below (SEQ ID No: 13) for proof of concept. This GC-rich DNA fragment contains similar amount of GC content (SEQ ID No: 13) (Table 3). It is, therefore, useful to test enhancer or “hot spot” activity when integrated into mammalian expression vectors.

A synthesized high GC content DNA fragment is listed below (SEQ ID No: 13):

GGGGGCTGCGGAGGAACAGAGAAGGGAAGATATAAACCCCGCCGGCG
CCGACGAACCCCGCCCTGCCCCGTCCCCCCCGAAGGCAGCCGTCCCC
CTGCGGCAGCCCCGAGGCTGGAGATGGAGAAGGGGACGGCGGCGCGG
CGACGCACGAAGGCCCTCCCCGCCCATTTCCTTCCTGCCGGCGCCGC
ACCGCTTCGCCCGCGCCCGCTAGAGGGGGTGCGGCGGCGCCTCCCAG
ATTTCGGCTCCGCCAGATTTGGGACAAAGGAAGTCCCTGCGCCCTCT
CGCACGATTACCATAAAAGGCAATGGCTGCGGCTCGCCGCGCCTCGA
CAGCCGCCGGCGCTCCGGGGCCGCCGCGCCCCTCCCCCGAGCCCTCC
CCGGCCCGAGGCGGCCCCGCCCCGCCCGGCACCCCCACCTGCCGCCA
CCCCCCGCCCGGCACGGCGAGCCCCGCGCCACGCCCCGCACGGAGCC
CCGCACCCGAAGCCGGGCCGTGCTCAGCAACTCGGGGAGGGGGGTGC
AGGGGGGGGTTACAGCCCGACCGCCGCGCCCACACCCCCTGCTCACC
CCCCCACGCACACACCCCGCACGCAGCCTTTGTTCCCCTCGCAGCCC
CCCCGCACCGCGGGGCACCGCCCCCGGCCGCGCTCCCCTCGCGCACA
CGCGGAGCGCACAAAGCCCCGCGCCGCGCCCGCAGCGCTCACAGCCG
CCGGGCAGCGCGGGCCGCACGCGGCGCTCCCCACGCACACACACACG
CACGCACCCCCCGAGCCGCTCCCCCCCGCACAAAGGGCCCTCCCGGA
GCCCTTTAAGGCTTTCACGCAGCCACAGAAAAGAAACGAGCCGTCAT
TAAACCAAGCGCTAATTACAGCCCGGAGGAGAAGGGCCGTCCCGCCC
GCTCACCTGTGGGAGTAACGCGGTCAGTCAGAGCCGGGGCGGGCGGC
GCGAGGCGGCGCGGAGCGGGGCACGGGGCGAAGGCAACGCAGCGACG
TCGAGCTGCAGCGGCCGATCCCTTCCTGGGACTGGCCATGGCCAACT
CACTTCTGAACCCCATCATCTACACGCTCACCAACCGCGACCTGCGC
CACGCGCTCCTGCGCCTGGTCTGCTGCGGACGCCACTCCTGCGGCAG
AGACCCGAGTGGCTCCCAGCAGTCGGCGAGCGCGGCTGAGGCTTCCG
GGGGCCTGCGCCGCTGCCTGCCCCCGGGCCTTGATGGGAGCTTCAGC
GGCTCGGAGCGCTCATCGCCCCAGCGCGACGGGCTGGACACCAGCGG
CTCCACAGGCAGCCCCGGTGCACCCACAGCCGCCCGGACTCTGGTAT
CAGAACCGGCTGCACTGCA

High GC content regions of this GC-rich DNA fragment (SEQ ID No: 13) was analyzed and summarized in Table 3 below.

TABLE 3

| Positions | 1-100 | 351-490 | 601-730 | 951-1100 | 1121-1335 |
|---|---|---|---|---|---|
| GC content | 73.0% | 88.6% | 85.4% | 68.7% | 73.0% |

By using this GC-rich DNA fragment (SEQ ID No: 13), we constructed pMH8 (pGC rich fragment-actin promoter-poly linker-polyA) (FIG. 9) (SEQ ID No:11) and pMH9 (pActin promoter-poly linker-poly A-GC rich fragment) (FIG. 10) (SEQ ID No:12) (see Example 2). Expression results were shown in EXAMPLE 4 and clearly indicated that its strong enhancer or “hot spot” activity similar to that of chick beta actin gene intron-1. We concluded that high GC-rich DNA sequence could be used to construct to reconstruct gene expression vectors as a common method for high gene expression. Possibly, it is a universal mechanism that governs all eukaryotic gene expression.

By the terminology “GC-rich fragment” as used throughout this description (unless otherwise specified), there is meant a piece of DNA (100-2000 bp in length), either naturally occurring or synthesized, in which not less than about sixty eight percent (68%) by number of the bases are composed of cytosine (C) and/or guanine (G), and most preferably, eighty percent (80%) or more by number are composed of cytosine and/or guanine.

### Example 4

### Expression of TNFR2-Fc to Compare Strength of the Expression Vectors

A cDNA encoding EcoRI site-TNFR2-Fc-Not I site (SEQ ID No 14) was removed form a previous plasmid vector (in house) and inserted into EcoRI/Not I sites of the above constructed mammalian expression vectors shown in FIG. 1-10 (SEQ ID No 3, 4, 5, 6, 7, 8, 9, 10, 11, 12). These plasmid cDNAs were linearized by PvuI and stably transfected into a fast growing CHO parental host line using a Gene Pulser (Bio-Rad). PGK promoter driven neomycin resistant gene was used for stable cell clone selection either through co-transfection or through inserting PGK-Neo resistant gene-pA cassette into SalI site of the each vector.

The stable cell clones were picked into a 96-well plate (NUNC). The transfection was repeated. All gene expressions were conducted in 0.1 ml freshly added serum-free medium at 37° C. in a CO₂ incubator in 96-well plate for 3 hours.

The TNFR2-Fc expression of 3 hours in fresh serum-free medium was detected by using a dot-blot or Elisa. Anti-human IgG1 Fc fragment antibodies conjugated with HRP (PIERCE) were used for the specific binding. Expression titer of the best clone from the above two transfections of 2×96-well plates was used to compare expression titer of each constructs.

In brief, the harvested conditional media were diluted seriously at 0, 2, 4, 8, 16, and 32 times. The diluted conditional media were subjected to dot blot semi-quantitative assay using anti human Ig Fc antisera conjugated with HRP (PIERCE). Alternatively, 96-well micro-plate for a standard Elisa was coated by using 0.1 ml of the diluted conditional media followed by incubating with anti human Ig Fc antisera conjugated with HRP (PIERCE), washing, color development and quantitation by a micro-plate reader. Commercial available TNFR2-Fc (Enbrel) was added to our serum-free culture medium and used as a quantitative standard.

The results below in Table 1 indicated that this 1.006 kb chick beta actin gene Intron-1 could be used as a gene expression enhancer element or gene expression “hot spot” sequence at 5- or 3′-flanking of a mammalian gene expression promoter to construct a new mammalian expression vector or modify an existed gene expression vector for high-level expression of recombinant proteins and generation of mammalian cell lines producing high level of recombinant proteins.

The results clearly indicated that the intron-1 is not only an enhancer element but also a “hot spot” sequence since it works well at all different locations of the expression vectors.

In addition, it showed that a synthetic GC-rich fragment also can be used as a gene expression enhancer element or gene expression “hot spot” sequence at 5′- or 3′-flanking of a mammalian gene expression promoter.

All the expression titers reached or exceeded high end of current industrial levels (15-45 pg/cell/day), suggesting great commercial value of these expression vectors. We believed that we had solved mammalian gene expression once for all and identified probably a common mechanism of all gene expression, namely use of naturally occurred or synthetic GC-rich DNAs with strong structure as enhancers or expression “hot spot” sequences for high constitutive mammalian gene expression.

TABLE 1

| | | # of | Expression titer |
|---|---|---|---|
| | | clones | (pg/cell/day) of |
| Vector | Figure/SEQ ID | screened | the best clone |
| Control | FIG. 1/(SEQ ID No: 3 | 96 × 2 | 7 ± 2 |
| pMH1 | FIG. 2/SEQ ID No: 4 | 96 × 2 | 53 ± 4 |
| pMH2 | FIG. 3/SEQ ID No: 5 | 96 × 2 | 52 ± 4 |
| pMH3 | FIG. 4/SEQ ID No: 6 | 96 × 2 | 67 ± 5 |
| pMH4 | FIG. 5/SEQ ID No: 7 | 96 × 2 | 56 ± 3 |
| pMH5 | FIG. 6/SEQ ID No: 8 | 96 × 2 | 60 ± 5 |
| pMH6 | FIG. 7/SEQ ID No: 9 | 96 × 2 | 69 ± 7 |
| pMH7 | FIG. 8/SEQ ID No: 10 | 96 × 2 | 45 ± 2 |
| pMH8 | FIG. 9/SEQ ID No: 11 | 96 × 2 | 41 ± 4 |
| pMH9 | FIG. 10/SEQ ID No: 12 | 96 × 2 | 39 ± 5 |

As we discussed earlier in this invention, plant gene 5′ regulatory regions often contain high GC-rich content called CpG islands. Plant gene expression is often constitutive at higher levels. The results in Table 1 indicated that a naturally occurred intron-1 of chick beta actin gene with extremely high GC-rich content and possible strong DNA structure played a key role for CHO cell gene expression. This indicated that searching for high GC content introns or expression enhancer or insulators for mammalian gene expression will be universal tool for constructing effective gene expression vectors. Other option is to synthesize artificial GC-rich introns, “shot spot”, enhancers, promoters for constructing and reconstructing effective gene expression vectors.

The results in Table 1 also indicated that integration of a non-specific synthetic GC-rich DNA fragments support high level of constitutive gene expression in CHO cells, suggesting future use of GC-rich DNA sequence for synthetic gene expression enhancer or “hot spot” as a universal tool for gene expression vector construction. Very likely, high GC-content DNA fragment with strong DNA structure is a universal mechanism that regulates chromatin condensation and nucleosome-formation for high level of gene transcription and expression.

### Example 5

### Promoter Strength Analysis of Control Vector and pMH4

The native chick beta actin promoter-based expression vector (FIG. 1) (SEQ ID NO: 3) somehow was not strong enough to serve commercial purpose although it contains the intron-1 (SEQ ID NO: 1). We thus analyzed its promoter sequence below:

### Chick Beta Actin Promoter Sequence

CTCGAGGTGAGCCCCACGTTCTGCTTCACTCTCCCCATCTCCCCCCC
CTCCCCACCCCCAATTTTGTATTTATTTATTTTTTAATTATTTTGTG
CAGCGATGGGGGCGGGGGGGGGGGGGGCGCGCGCCAGGCGGGGCGGG
GCGGGGCGAGGGGCGGGGCGGGGCGAGGCGGAGAGGTGCGGCGGCAG
CCAATCAGAGCGGCGCGCTCCGAAAGTTTCCTTTTATGGCGAGGCGG
CGGCGGCGGCGGCCCTATAAAAAGCGAAGCGCGCGGCGGGCGGGAGT
CGCTGCGTTGCCTTCGCCCCGTGCCCCGCTCCGCGCCGCCTCGCGCC
GCCCGCCCCGGCTCTGACTGACCGCGTTACTCCCACAG

It contains only one TATA box and two transcription factor binding site CAAT boxes. Clearly, it is not a typical strong promoter. We therefore replace the actin promoter with a typical CMV promoter (pMH4) (FIG. 5) (SEQ ID NO: 7). Sequence of CMV promoter used is listed below for analysis.

CMV Promoter Sequence

ACGCGTCGACGGATCGGGAGATCTCCCGATCCCCTATGGTGCACTCT
CAGTACAATCTGCTCTGATGCCGCATAGTTAAGCCAGTATCTGCTCC
CTGCTTGTGTGTTGGAGGTCGCTGAGTAGTGCGCGAGCAAAATTTAA
GCTACAACAAGGCAAGGCTTGACCGACAATTGCATGAAGAATCTGCT
TAGGGTTAGGCGTTTTGCGCTGCTTCGCGATGTACGGGCCAGATATA
CGCGTTGACATTGATTATTGACTAGTTATAGTAATCAATTACGGGGT
CATTAGTTCATAGCCCATATATGGAGTTCCGCGTTACATAACTTACG
GTAAATGGCCCGCCTGGCTGACCGCCCAACGACCCCCGCCCATTGAC
GTCAATAATGACGTATGTTCCCATAGTAACGCCAATAGGGACTTTCC
ATTGACGTCAATGGGTGGAGTATTTACGGTAAACTGCCCACTTGGCA
GTACATCAAGTGTATCATATGCCAAGTACGCCCCCTATTGACGTCAA
TGACGGTAAATGGCCCGCCTGGCATTATGCCCAGTACATGACCTTAT
GGGACTTTCCTACTTGGCAGTACATCTACGTATTAGTCATCGCTATT
ACCATGGTGATGCGGTTTTGGCAGTACATCAATGGGCGTGGATAGCG
GTTTGACTCACGGGGATTTCCAAGTCTCCACCCCATTGACGTCAATG
GGAGTTTGTTTTGGCACCAAAATCAACGGGACTTTCCAAAATGTCGT
AACAACTCCGCCCCATTGACGCAAATGGGCGGTAGGCGTGTACGGTG
GGAGGTCTATATAAGCAGAGCTCTCTGGCTAACTAGAGAACCCACTG
CTTACTGGCTTATCGAAATTAATACGACTCACTATAGGGAGACCCAA
GCTGGCTAGCGTTTAAACTCTGCAGAACCAATGCATTGGAT

Two TATA boxes and ten CAAT boxes are discovered. Not only numbers of CAAT boxes increased when compared with the actin promoter, but also distance between these CAAT boxes and GC-rich intron-1 region increased. The increased distance might make transcription factor binding more efficient by avoiding GC-rich intron-1 formed strong structure.

Table-1 shows 8-time increase of gene expression. This suggested that chick beta actin promoter was somehow mutated to current strength during evolution process even though it contains the strongest enhancer element namely intron-1 known up to date. Use of isolated chick beta actin intron-1 from full length of beta actin gene promoter is a key for construction and reconstruction of mammalian expression vectors for production of recombinant proteins.

### Example 6

### Use of at the 3′ Flanking Region Poly A Site

Addition intron-1 at the 3′ flanking region of poly A site (pMH3) (FIG. 4) increased gene expression significantly when compared with control (Table-1). This intron-1 location is far away from actin promoter sequence as there is a recombinant TNFR2-Fc coding gene and poly a sequence in between. Most likely, the intron-1 is not only an enhancer element but also a “hot spot” sequence. It increases the gene expression level through its GC-rich DNA structure, which opens genomic DNA structure or chromatin to increase accessibility of nuclear transcription factors.

1. An expression vector for use in the recombinant production of a polypeptide in a mammalian cell, which comprises (a) a mammalian promoter sequence, (b) a DNA sequence encoding a recombinant polypeptide, (c) a poly A site, and (d) a GC-rich DNA fragment which enhances expression of the polypeptide.

2. The expression vector of claim 1 in which the GC-rich fragment is fused to the 5′ flanking region of the mammalian promoter sequence.

3. The expression vector of claim 1 in which the GC-rich fragment is fused to the 3′ flanking region of the mammalian promoter sequence.

4. The expression vector of claim 1 in which the GC-rich fragment is fused to the 3′ flanking region of a poly A site of a mammalian expression vector.

5. A method for the recombinant production of a polypeptide, comprising expressing the polypeptide in a mammalian cell in conditions of high density cell growth under the control of an expression vector which comprises (a) a mammalian promoter sequence, (b) a DNA sequence encoding a recombinant polypeptide, (c) a poly A site, and (d) a GC-rich DNA fragment which enhances expression of the polypeptide.

6. The method of claim 5 in which the GC-rich fragment of the expression vector is fused to the 5′ flanking region of the mammalian promoter sequence.

7. The method of claim 5 in which the GC-rich fragment of the expression vector is fused to the 3′ flanking region of the mammalian promoter sequence.

8. The method of claim 5 in which the GC-rich fragment is fused to the 3′ flanking region of a poly A site of a mammalian expression vector.

9. A method for improving the effectiveness of a gene expression vector which comprises including in the vector a chick beta actin intron 1 or functional equivalent thereof.

10. The method of claim 9 in which the functional equivalent of the chick beta actin intron 1 is a GC-rich fragment.

11. An expression vector for use in the recombinant production of a polypeptide in a mammalian cell, which comprises (a) a chick beta actin intron 1, or functional equivalent thereof, fused to the flanking region of a mammalian promoter sequence, (b) a gene sequence encoding a recombinant polypeptide, (c) a poly A site, (d) a chick beta actin intron 1, or functional equivalent thereof, and (e) a pBR322 vector backbone.

12. The expression vector of claim 11 in which the functional equivalents for elements (a) and (d) are GC-rich DNA fragments.

13. The expression vector of claim 11 in which the chick beta actin intron 1 of element (a), or functional equivalent, is fused to the 5′ flanking region of a mammalian promoter sequence.

14. The expression vector of claim 11 in which the chick beta actin intron 1 of element (a), or functional equivalent, is fused to the 3′ flanking region of a mammalian promoter sequence or downstream of poly A sequence.

15. The expression vector of claim 11 in which the chick beta actin intron 1 of element (a), or functional equivalent, is fused to the 3′ flanking region of a poly A site of a mammalian expression vector.

16. The expression vector of claim 11, which includes the sequence of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

17-24. (canceled)

25. A method for the recombinant production of a polypeptide, comprising expressing the polypeptide in a mammalian cell in conditions of high density cell growth under the control of an expression vector comprising comprises (a) a chick beta actin intron 1, or functional equivalent thereof, fused to the flanking region of a mammalian promoter sequence, (b) a gene sequence encoding a recombinant polypeptide, (c) a poly A site, (d) a chick beta actin intron 1, or functional equivalent thereof, and (e) a pBR322 vector backbone.

26. The method of claim 25 in which the functional equivalents for elements (a) and (d) are GC-rich DNA fragments.

27. The method of claim 25 in which the chick beta actin intron 1 of element (a), or functional equivalent, is fused to the 5′ flanking region of the mammalian promoter sequence of the expression vector.

28. The method of claim 25 in which the chick beta actin intron 1 of element (a), or functional equivalent, is fused to the 3′ flanking region of the mammalian promoter sequence for the expression vector.

29. The method of claim 25 in which the chick beta actin intron 1 of element (a), or functional equivalent, is fused to the 3′ flanking region of a poly A site of a mammalian expression vector.

30. The method of claim 25 in which the expression vector includes the sequence of SEQ ID NO: 4., SEQ ID NO. 5, SEQ ID NO. 6, SEQ ID NO. 7, SEQ ID NO. 8, SEQ ID NO. 9, SEQ ID NO. 10, SEQ ID NO. 11 AND SEQ ID NO. 12.

31-38. (canceled)

39. A method for enhancing the performance of an existed expression vector for use in the recombinant production of a polypeptide in a mammalian cell, comprising introducing in said vector the chick beta actin intron 1, or functional equivalent thereof, at either flanking region of an existing promoter or poly A site.
